(19) United States
(12) Patent Application Publication
(10) Pub. No.: US 2008/0058905 A1
(43) Pub. Date: Mar. 6, 2008

(54) METHOD AND APPARATUS UTILIZING LIGHT AS THERAPY FOR FUNGAL INFECTION

(76) Inventor: Darrell O. Wagner, Isanti, MN (US)

Correspondence Address: SCHWEGMAN, LUNDBERG & WOESSNER, P.A., P.O. BOX 2938, MINNEAPOLIS, MN, 55402, US

(21) Appl. No.: 11/847,539

(22) Filed: Aug. 30, 2007

Related U.S. Application Data
(60) Provisional application No. 60/824,381, filed on Sep. 1, 2006.

(51) Int. Cl. A61N 5/06

(52) U.S. Cl. 607/88

(57) ABSTRACT

Described herein are methods and apparatus for treating fungal nail infections and similar diseases with light therapy. In one embodiment, an apparatus that utilizes one or more blue light emitting diodes (LED) to irradiate fungus residing under and around the nail is applied externally to a toe or finger in order to kill the fungus and restore normal nail growth. Light therapy may applied in this manner periodically at scheduled times. Another embodiment involves the use of a bootie-like structure having blue light emitting diodes for irradiating the toes, heal, and foot bottom.

### CLAIM OF PRIORITY

This application claims the benefit under 35 U.S.C. 119(e) of U.S. Provisional Patent Application Ser. No. 60/824,381, filed Sep. 1, 2006, the entire disclosure of which is hereby incorporated by reference in its entirety.

### FIELD OF THE INVENTION

This invention pertains to methods and devices for treating cutaneous disease.

### BACKGROUND

Fungal infection, especially of the toes and feet is a common problem. One such fungus, tinea pedis, causes athelete's foot. Fungal infections may also occur in the nail bed, matrix, or nail plate of fingers and toes. The medical terms for this type of fungal infection are onychomycosis or tinea unguium. Fungal infections of the nail are due to many factors and may affect a significant portion of the population in developed countries. The most common type of fungal nail infection involves the end of the nail when the fungi invade the hyponychium. Initially, the nail plate splits from the nail bed, a process called onycholysis. The end of the nail then turns yellow or white, and keratin debris develops under the nail causing further separation. The fungus grows in the substance of the nail, causing it to become fragile and crumble. The fungal organism responsible for most fungal nail infections is trichophyton rubrum.

Once the fungus establishes itself under a toenail or fingernail, it is difficult to cure. Topical preparations are not usually effective in treating fungal nail infections. The only generally effective approach involves oral medications that enter the nail from the blood. All of these medications have significant side effects and interact with many other medications. Anyone taking oral antifungal medications must have periodic tests done to monitor liver and blood cell function. The medications are also expensive and must be taken for several months.

### SUMMARY

Described herein are methods and apparatus for treating fungal nail infections and similar diseases with light therapy. In one embodiment, an apparatus that utilizes one or more blue light emitting diodes (LED) to irradiate fungus residing under and around the nail is applied externally to a toe or finger in order to kill the fungus and restore normal nail growth. Light therapy may be applied in this manner periodically at scheduled times. Another embodiment involves the use of a bootie-like structure having blue light emitting diodes for irradiating the toes, heal, and foot bottom.

### BRIEF DESCRIPTION OF THE DRAWINGS

FIGS. 1 through 3 illustrate different embodiments of apparatus for delivery cutaneous light therapy.

### DETAILED DESCRIPTION

Research has demonstrated the effectiveness of blue light at approximately 470 m wavelength in causing retarded growth and cell death. Such effects have been found to occur with light exposure time periods of between 8 and 72 hours. Described herein are apparatus having one or more blue light LEDs that are adapted to kill fungus residing on the body on such areas as the fingers, toes and feet. The blue light LEDs may be designed to emit light at a wavelength of 470 nm or within a range of 400 to 500 nm and with an intensity of 7500 mcd or within a range of 1000 to 10000 mcd.

One embodiment includes a clip designed to fit over toe or finger in a manner similar to clips worn on the finger for use in pulse oximetry. The clip in this embodiment is a toe or finger clip that includes two hinging portions with a spring to keep the clip closed around the toe or finger. The clip may contain one or more blue light LEDs mounted in the clip light is emitted into the nail when the clip is worn. The apparatus may include a battery, an on/off switch, and control circuitry for controlling the operation of the LEDs, all or part of which may be incorporated into the clip or be located externally.

Another embodiment includes a bootie that may be worn on the foot while sitting or laying down such as during sleep. The bootie may contain an array of blue LEDs across the front, back and bottom facing towards the inserted foot. The bootie may also incorporate a clear foam-like padding between the LEDs and foot for providing comfort. The apparatus may include a battery, an on/off switch, and control circuitry for controlling the operation of the LEDs, all or part of which may be incorporated into the bootie or be located externally. In one embodiment, power for the LEDs within the bootie is provided by batteries housed on a cuff worn around the ankle above the foot needing treatment, and a wire cable connects the cuff to the bootie. The cuff in this embodiment may also have an on/off switch.

Other embodiments include a disposable clip or substrate containing blue LEDs that can be fixed to the area needing treatment, where fixation may be performed with an adhesive. Power, switch and control circuitry in this embodiment may be provided by a separate module worn by the person on the ankle or wrist.

In any of the apparatus for delivering light therapy as described herein, a timer may be incorporated into the control circuitry of the apparatus to limit the light exposure time. The timer may be controlled by a user interface that allows a patient to manually set the time for which light is to be delivered. A predetermined exposure time, such as eight hours, may be programmed in the timer. In certain cases, only one eight hour treatment may be necessary. Time is then allowed for new nail material to grow and the old nail with deceased fungus to be removed.

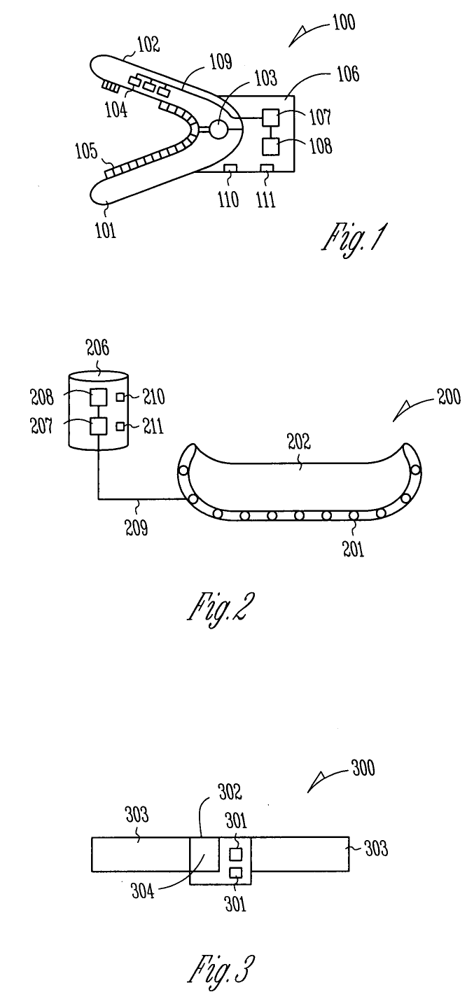

FIG. 1 depicts one embodiment of an apparatus for delivering cutaneous light therapy to a toenail or fingernail. A clip 100 comprises a lower portion 101 and an upper portion 102 that are opposed to one another and each pivotally attached to a spring 103. The two portions may be pivoted apart around the spring in order to open the clip, while the spring acts to hinge the two portions together and close the clip around a toe or finger. The upper portion of the clip incorporates one or more blue LEDs 104 disposed so as to emit light toward the nail when the clip is closed around a toe or finger. A foam padding 105 may be fitted over the inner surfaces of the upper and lower clip portions for patient comfort. Mounted within a body portion 106 of the clip are control circuitry 107 and a battery 108 which connect to the LEDs by wires 109. The control circuitry controls operation of the LEDs by gating power from the battery. Interfaced to the control circuitry is an on-off switch 110 that may be actuated by the patient. Timing circuitry may also be incorporated into the control circuitry to allow therapy to be delivered for predetermined periods. A timer switch 111 may be provided to allow the patient to set a specified duration for the therapy.

FIG. 2 shows another embodiment of an apparatus for delivering cutaneous light therapy that includes a bootie 200. This embodiment may be used to treat not only nail infections but other cutaneous infections as well such as athlete's foot. The bootie 200 is a contoured structure into which may be fitted a patient's foot. The bootie 200 incorporates one or more blue LEDs 201 disposed on its inner surface so as to radiate toward the patient's skin. A liner 202 made of clear foam material may be interposed between the LEDs and the patient's skin to enhance patient comfort while still allowing light transmission. The apparatus also includes a cuff 206 that may be worn around the patient's arm or ankle. Mounted within the cuff 206 are control circuitry 207 and a battery 208 which connect to the LEDs by wires 209. The control circuitry controls operation of the LEDs by gating power from the battery. Interfaced to the control circuitry is an on-off switch 210 that may be actuated by the patient. Timing circuitry may also be incorporated into the control circuitry to allow therapy to be delivered for predetermined periods. A timer switch 211 may be provided to allow the patient to set a specified duration for the therapy.

FIG. 3 shows another embodiment that includes a substrate 300 that incorporates one or more blue LEDs 301. The substrate 300 includes a midportion 302 for containing the LEDs 301 which are disposed so as to radiate toward the patient's skin. The substrate 300 also includes peripheral portions 303 which are adapted for fixation to the patient's skin such as by an adhesive or by being wrapped around an extremity and then mechanically connected together (e.g., by a hook and loop fastener). In one embodiment, the LEDs are connected to an external control device such as the cuff 206 described above which contains the battery and control circuitry. In another embodiment, the midportion 302 has a compartment for containing the LEDs and battery 304. The apparatus may be constructed so that the LEDs are activated when inserted into the midportion with the battery. In this embodiment, the substrate may be made disposable after removal of the battery and LEDs.

Although the invention has been described in conjunction with the foregoing specific embodiment, many alternatives, variations, and modifications will be apparent to those of ordinary skill in the art. Such alternatives, variations, and modifications are intended to fall within the scope of the following appended claims.

What is claimed is:

1. An apparatus for treating cutaneous fungal infections, comprising:
a structure for fitting over a patient's extremity;
one or more blue LEDs incorporated into the structure for emitting light toward the patient's skin;
control circuitry for controlling operation of the LEDs; and,
a timer incorporated into the control circuitry controlled by a user interface that allows a patient to manually set the time for which light is to be delivered.

2. The apparatus of claim 1 wherein the structure is a toe or finger clip that includes two hinging portions with a spring to keep the clip closed around a toe or finger, the clip containing one or more blue light LEDs mounted in the clip that emit light into a nail of the toe or finger when the clip is worn.

3. The apparatus of claim 2 wherein the clip comprises a lower portion and an upper portion that are opposed to one another and each pivotally attached to a spring, wherein the two portions may be pivoted apart around the spring in order to open the clip with the spring acting to hinge the two portions together and close the clip around a toe or finger.

4. The apparatus of claim 3 wherein the upper portion of the clip incorporates one or more blue LEDs disposed so as to emit light toward a nail when the clip is closed around a toe or finger.

5. The apparatus of claim 2 further comprising a foam padding that may be fitted over the inner surfaces of the upper and lower clip portions for patient comfort.

6. The apparatus of claim 1 wherein the structure is a bootie that may be worn on the foot, the bootie containing an array of blue LEDs across the front, back and bottom facing towards the inserted foot.

7. The apparatus of claim 6 wherein the bootie incorporates a clear foam-like padding between the LEDs and foot for providing comfort.

8. The apparatus of claim 6 further comprising a cuff worn around the ankle above the foot for containing batteries to supply power to the LEDs within the bootie and a wire cable connecting the cuff to the bootie.

9. The apparatus of claim 1 wherein the structure is a disposable substrate containing blue LEDs that incorporates an adhesive for fixing the substrate to an area needing treatment.

10. The apparatus of claim 6 further comprising a separate module for wearing on the ankle or wrist for containing power and control circuitry.

11. A method for treating cutaneous fungal infections, comprising:
fitting a structure for fitting over a patient's extremity, wherein the structure incorporates one or more blue LEDs for emitting light toward a patient's skin;
emitting light from the LEDs at a wavelength between 400 and 500 nm; and,
setting a timer for controlling operation of the LEDs so that the LEDs emit light for a specified period of time.

12. The method of claim 11 wherein the structure is a toe or finger clip that includes two hinging portions with a spring to keep the clip closed around a toe or finger and further comprising fitting the clip over a toe or finger so that the LEDs mounted in the clip emit light into a nail of the toe or finger.

13. The method of claim 12 further comprising fitting a foam padding over the inner surfaces of the upper and lower clip portions.

14. The method of claim 1 wherein the structure is a bootie and further comprising fitting the bootie over a patient's foot so that the LED's emit light thereto.

15. The method of claim 14 further comprising providing a clear foam-like padding between the LEDs and foot.

16. The method of claim 14 further comprising fitting a cuff worn the ankle above the foot, wherein the cuff contains batteries to supply power to the LEDs within the bootie, and connecting the cuff to the bootie with a wire cable.

17. The method of claim 11 wherein the structure is a disposable substrate containing blue LEDs further comprising fixing the substrate to an area needing treatment with an adhesive.

18. The method of claim 17 further comprising fitting a separate module on the ankle or wrist, wherein the separate module contains power and control circuitry for the LEDs of the substrate.
